(19) United States
(12) Patent Application Publication
(10) Pub. No.: US 2026/0240745 A1
(43) Pub. Date: Aug. 20, 2026

(54) INDIGO-BASED DYE WITH IMPROVED STABILITY AND IMPROVED DYEING PROPERTIES

(71) Applicant: HENKEL AG & CO. KGAA, Düsseldorf (DE)

(72) Inventors: Skrollan KLAAS, Hamburg (DE); Jana Marie ROESEKE, Tornesch (DE); Nele DALLMANN, Hamburg (DE); Fabian STRASKE, Hamburg (DE); Thomas HIPPE, Appen (DE)

(21) Appl. No.: 19/161,717

(22) PCT Filed: Mar. 8, 2024
(86) PCT No.: PCT/EP2024/056124

§ 371 Date: Sep. 3, 2025

(30) Foreign Application Priority Data
Mar. 13, 2023 (DE) 10 2023 202 220.6

(51) Int. Cl. A61K 8/66; A61K 8/44; A61K 8/49; A61Q 5/10

(52) U.S. Cl. CPC A61K 8/66; A61K 8/447; A61K 8/492; A61K 8/498; A61Q 5/10; A61K 2800/882

(57) ABSTRACT

A product, a kit, and a method for dyeing keratin fibers, such as human hair, uses an indican-containing component that was pretreated at a temperature in the range of 60° C. to 200° C., an enzyme mixture having cellulase activity, cysteine, and isatin.

The invention relates to agents, kits and methods for dyeing keratin fibers, in particular human hair, using an indican-containing component that was pretreated at a temperature in the range of 60° C. to 200° C., an enzyme mixture with cellulase activity, cysteine and isatin.

The desire to change one's hair color is a great need for many consumers. To satisfy this need, the cosmetic industry provides a diverse range of products. Hair dyes that achieve particularly long-lasting coloring with high coverage are usually oxidation dyes. These use oxidizing agents, which can damage the hair structure. Certain cationic direct azo dyes are also capable of producing hair color changes with excellent fastness properties. However, said azo dyes are synthetic dyes.

There is a demand among a growing number of consumers for hair dyes and hair dyeing methods that are based on natural dyes, even though these dyes and methods are often inferior to the aforementioned dyes and methods in terms of fastness properties, coverage, and color variety.

In addition to dyeing with henna, which is derived from the Lawsonia inermis plant, dyeing hair with plants from which indigo or indirubin can be produced has also been known for a long time.

### PRIOR ART

The preparation of storage-stable cosmetic products containing natural plant material is often difficult. The plant material contains enzymes that can react with other ingredients of the plant material or the cosmetic, including during storage. There has therefore been no shortage of attempts in the prior art to increase the storage stability of cosmetics containing plant materials. When dyeing with indigo- or indirubin-producing plant materials, it is often observed that the color changes continuously over a period of up to 2 weeks after application of the dye and that the final color tone only remains after this period. Starting from a concentration of 2 wt. % of indigo- or indirubin-producing plant powder in water, the color tone on the hair changes in the first few days after dyeing from an initial turquoise-blue tone to a redder and darker tone, creating a purple color impression.

There has been no lack of efforts in the prior art to improve the dyeing of keratin fibers, in particular human hair, with indigo.

US20040055096A1 discloses a dye for the preparation of which powdered leaves of an indigo-producing plant, such as Polygonum tinctorium, Strobilanthes cusia, Indigofera tinctoria or Isatis indigotica, whose natural enzyme content has been deactivated by heat treatment, are mixed, shortly before use, with a component containing beta-glucosidase (e.g. fresh leaves of an indigo-producing plant that have not been heat treated, or the pressed juice from these non-heat-treated leaves, possibly in freeze-dried form, or extracts of various mushrooms, such as enokitake (Flammulina velutipes) and shiitake (Lentinus Edodes), or extracts of corn leaves, apricot kernels or almonds). By separating the indigo precursor indican, which is present in the plants in the form of 3-indoxyl-β-D-glucopyranoside, from the plant's own enzyme beta-glucosidase, improved storage stability of the natural dye is achieved. PCT application WO2014104301A1 discloses a similar approach to improved storage stability, namely the combination of two different indigo powders, wherein the natural enzyme content in the first leaf powder component was deactivated by heat treatment, while the second leaf powder component was dried at temperatures below 50° C. and thus contained active enzyme.

WO2015082482 A1 discloses a method for dyeing hair using powdered plant parts from plants that produce indigo, in which the hair is post-treated with a strongly alkaline composition after being dyed with indigo, which can be carried out in the presence of hydrogen peroxide. This is intended to accelerate the development of the final color tone and to prevent any change in color after the actual dyeing process has been completed. However, the hair is damaged by the alkaline pH and the oxidizing agent.

DE4211450A1 discloses non-oxidative hair dyes which contain isatin in combination with at least one amino acid, for example cysteine, or a water-soluble oligopeptide composed of 2 to 9 amino acids in an aqueous carrier.

### OBJECT

One object was to provide agents, kits and methods for non-oxidatively dyeing keratin fibers, in particular human hair, using plants producing indigo or indirubin, with which a wider range of colors can be achieved. In particular, colors beyond the typical indigo blue, especially colors with a shift into warmer color ranges, should be provided. Another object of the present invention was to provide agents, kits and methods for non-oxidatively dyeing keratin fibers, in particular human hair, using plants that contain indican and produce indigo or indirubin, by means of which plants the final color result of dyeing with indigo is obtained as quickly as possible after completion of the actual dyeing process.

Another object of the present invention was to provide agents, kits and methods for non-oxidatively dyeing keratin fibers, in particular human hair, using plants containing indican and producing indigo or indirubin, with improved storage stability.

Surprisingly, it was found that, using parts or extracts of plants containing indican and producing indigo or indirubin in combination with cysteine and isatin, the dyes, dyeing methods and dyeing kits described in the claims could be used to achieve an intense magenta hair color immediately after the dyeing procedure. It was also possible that the final color result was already achieved shortly after completion of the actual dyeing process and that this color result-unlike what is often typical for dyeing with indigo-did not change any further in the days following completion of the dyeing process. Neither the initial turquoise-blue color tone characteristic of indigo nor the final purple tone were formed on the hair fiber. The dyes and dye kits according to the invention also have high storage stability.

The present invention thus first relates to an agent for non-oxidatively dyeing keratin fibers, in particular human hair, containing
i) at least one indican-containing component that was pretreated at a temperature in the range of 60° C. to 200° C., and
ii) an enzyme mixture comprising at least one endo-1,4-beta-glucanase (EC 3.2.1.4), at least one beta-glucosidase (EC 3.2.1.21), and at least one exo-1,4-beta-D-glucanase (EC 3.2.1.91), and
iii) cysteine, and
iv) isatin, and
v) water.

According to the invention, non-oxidative dyeing is understood to mean that the dyeing process takes place without the addition of oxidizing agents other than atmospheric oxygen.

The indigo precursor or indirubin precursor indican occurs naturally in plants. Indican is a glucoside of indoxyl and occurs naturally in some plants known as indigo plants, for example Isatis tinctoria (woad) or Indigofera tinctoria. Synonyms for indican are 3-indoxyl-β-D-glucopyranoside, indoxyl-β-D-glucoside, 3-(β-D-glucosido) indole or plant indican.

Before the indican is converted into the dyes indigo or indirubin, the indican must be split into indoxyl and the glucoside functional group. This cleavage process preferably occurs enzymatically. The cleavage process should occur in situ, i.e. just before application to the keratin fibers to be dyed.

Accordingly, the indican-containing component, which has been pretreated at a temperature in the range of 60° C. to 200° C., and the enzyme mixture ii) according to the invention should be physically separated from one another until they are used.

The present invention therefore also relates to a kit for non-oxidatively dyeing keratin fibers, in particular human hair, with natural dyes, which kit comprises the following compartments that are physically separated from one another:
i) a composition (Ind) containing at least one indican-containing component which has been pretreated at a temperature in the range of 60° C. to 200° C., and
ii) an aqueous composition (E) containing an enzyme mixture comprising at least one endo-1,4-beta-glucanase (EC 3.2.1.4), at least one beta-glucosidase (EC 3.2.1.21), and at least one exo-1,4-beta-D-glucanase (EC 3.2.1.91),
iii) optionally at least one further compartment,
   and which is characterized in that at least one of the compartments contains cysteine and at least one of the compartments contains isatin.

According to the invention, “non-oxidative” is understood to mean that no oxidizing agents other than atmospheric oxygen are used in the agents, kits and methods according to the invention. In particular, the oxidizing agents hydrogen peroxide, persulfates, perbromates, percarbonates, perborates and percarbamides are excluded. Atmospheric oxygen is not an oxidizing agent in the context of the invention.

Preferred agents and kits for non-oxidative dyeing according to the invention are characterized in that the indican-containing component i) is selected from crushed parts of an indigo- or indirubin-producing plant and from indican-containing extracts of an indigo- or indirubin-producing plant and mixtures of these indican-containing components i).

Particularly preferred agents and kits for non-oxidative dyeing according to the invention are characterized in that the indican-containing component i) is selected from indican-containing extracts of an indigo- or indirubin-producing plant.

Further agents and kits for non-oxidative dyeing that are preferred according to the invention are characterized in that the extraction agent used to obtain the at least one indican-containing extract from an indigo- or indirubin-producing plant is selected from water and C₁-C₄ alkanols and C₂-C₄ polyols, in particular ethanol, isopropanol, n-propanol, ethylene glycol, 1,2-propanediol, glycerin and 1,3-butylene glycol, as well as mixtures of these extraction agents, preferably selected from water, ethanol, mixtures of water and at least one C₁-C₄ alkanol, mixtures of water and at least one C₂-C₄ polyol and water-ethanol mixtures, particularly preferably selected from water, ethanol and water-ethanol mixtures.

Preferred agents and kits for non-oxidative dyeing according to the invention are characterized in that the indigo- or indirubin-producing plant(s) is/are selected from at least one species of the following genera:
Indigofera, in particular Indigofera tinctoria, Indigo suffruticosa, Indigofera articulata, Indigofera arrecta, Indigofera heterantha “Gerardiana”, Indigofera argentea or Indigofera longiracemosa;
Isatis, in particular Isatis tinctoria (woad);
Persicaria, in particular Persicaria tinctoria (dyer's knotweed);
Wrightia, in particular Wrightia tinctoria;
Calanthe, in particular Calanthe veratrifolia; and
Baphicacanthus cusia, synonym Strobilanthes cusia,
Plants producing indigo or indirubin that are particularly preferred according to the invention are selected from the genus Indigofera and in particular from Indigofera tinctoria.

The plants of the genera and species mentioned above contain no or only small amounts of indigo or indirubin. Instead, the plants contain indican, which is a precursor of indigo after enzymatic cleavage of the glucoside functional group. The parts of the above-mentioned plant genera and species that are rich in indican and also contain indigo and indirubin are the leaves of these plants. In order to obtain the indican-containing component i) according to the invention, the plant leaves of the above-mentioned plant genera and species are dried and ground and used as plant powder.

Plant powders that are preferred according to the invention have a particle size of less than 500 μm, particularly preferably in the range of 120 μm-200 μm, extremely preferably in the range of 150 μm-180 μm. Other plant powders that are preferred according to the invention have a bulk density in the range of 0.20-0.60 g/cm³, particularly preferably in the range of 0.25-0.40 g/cm³. Further plant powders preferred according to the invention have, relative to the weight thereof, a moisture content of less than 10 wt. %, preferably of 0.1 to 6.0 wt. %, particularly preferably of 0.2 to 3.0 wt. %. The moisture content is determined after 3 hours of drying at 105° C.

It is preferred according to the invention that the powder of the indigo- or indirubin-producing plant consists, based on its weight, of at least 50 wt. %, preferably at least 80 wt. %, particularly preferably more than 80-100 wt. %, of the leaves of the plant. Further embodiments that are preferred according to the invention are characterized in that the powder of the indigo- or indirubin-producing plant has a particle size of less than 500 μm, particularly preferably in the range of 120 μm-200 μm, extremely preferably in the range of 150 μm-180 μm, and a bulk density in the range of 0.20-0.60 g/cm³, particularly preferably in the range of 0.25-0.40 g/cm³, and has, based on its weight, a moisture content of less than 10 wt. %, preferably 0.1-6.0 wt. %, particularly preferably 0.2-3.0 wt. %.

Further agents and kits that are preferred according to the invention are characterized in that the powder of the indigo- or indirubin-producing plant contains indican in an amount of 2-5 wt. %, preferably 2.5-4.5 wt. %, particularly preferably 3-4.1 wt. %, based on the weight of the powder, wherein the indican content is converted to pure indican.

### Deactivation of the Natural Enzyme Content in the Indican-Containing Component i)

Further agents and kits for non-oxidative dyeing that are preferred according to the invention are characterized in that the at least one indican-containing component i) is pretreated at a temperature in the range from 60° C. to 200° C. by heating, in particular by heating in a solvent, furthermore by treatment with steam or by dry heating, preferably for a period of 1 to 120 minutes, particularly preferably 2 to 60 minutes, extremely preferably 5 to 10 minutes.

### Option 1: Indican-Containing Component i) is an Extract

Agents and kits for non-oxidative dyeing that are particularly preferred according to the invention are characterized in that the indican-containing component i) is selected from at least one indican-containing extract of an indigo- or indirubin-producing plant.

Agents and kits for non-oxidative dyeing that are extremely preferred according to the invention are characterized in that the indican-containing component i) is selected from at least one indican-containing extract of an indigo- or indirubin-producing plant, wherein extraction agent used to obtain the at least one indican-containing extract from an indigo- or indirubin-producing plant is selected from water and C₁-C₄ alkanols and C₂-C₄ polyols, in particular ethanol, isopropanol, n-propanol, ethylene glycol, 1,2-propanediol, glycerin and 1,3-butylene glycol, as well as mixtures of these extraction agents, preferably selected from water, ethanol, mixtures of water and at least one C₁-C₄ alkanol, mixtures of water and at least one C₂-C₄ polyol and water-ethanol mixtures, particularly preferably selected from water, ethanol and water-ethanol mixtures.

The indican can be extracted using these aforementioned polar solvents. Since the indican can be easily extracted at a temperature in the range of 60° C. to 200° C. by heating the solvent, this preferred procedure not only isolates the indican, but also deactivates the plant's own enzymes at the same time. The deactivation of the plant's own enzymes is desirable according to the invention, because it was surprisingly found that the coloration achieved can be significantly improved if the plant's own enzymes, which as a natural product are subjected to a multitude of qualitative and quantitative fluctuations in composition, are replaced by a standardized enzyme mixture.

In a further preferred embodiment of the invention, the extracted indican is not separated from the solvent after completion of the extraction procedure and separation of the solid plant parts, for example by filtration or decanting, but is directly used as the indican-containing component i), optionally after concentration by evaporation of the solvent or after dilution, wherein optionally further cosmetic aids, such as preservatives, thickeners, perfumes, pH regulators or hair conditioners, can be added.

In another, likewise preferred embodiment of the invention, the extracted indican is, for example, also separated from the solvent after completion of the extraction procedure and separation of the solid plant parts, for example by filtration or decanting, by freeze-drying or by distilling off the solvent such that the indican-containing component i) is in powder form. To prepare the dye preferred according to the invention, the powdered indican-containing component i) can be taken up in water and then mixed with the enzyme mixture and optionally additional water. In a further preferred embodiment, to prepare the dye preferred according to the invention, the powdered indican-containing component i) can be mixed with the enzyme mixture and with water.

### Option 2: Dried and Crushed Plant Parts of an Indigo-Producing Plant as the Indican-Containing Component i)

According to the invention, dried and crushed plant parts of an indigo-producing plant which have been pretreated at a temperature in the range of 60° C. to 200° C. can also be used as the indican-containing component i).

The heat pretreatment in this embodiment of the invention is characterized in that the at least one indican-containing component i) is pretreated at a temperature in the range from 60° C. to 200° C. by heating, in particular by heating without solvent, furthermore by treatment with steam or by dry heating, preferably for a period of 1 to 120 minutes, particularly preferably 2 to 60 minutes, extremely preferably 5 to 10 minutes.

Further dyes that are preferred according to the invention are characterized in that dried and crushed parts of at least one indigo- or indirubin-producing plant, which have been pretreated at 60-200° C., are contained as the at least one indican-containing component i) in a total amount of 1-80 wt. %, preferably 2-50 wt. %, particularly preferably 3-25 wt. %, extremely preferably 4-15 wt. %, further extremely preferably 5-10 wt. %, further extremely preferably 6-7 wt. %, based on the weight of the agent.

Further dyes that are preferred according to the invention are characterized in that powdered leaves of Indigofera tinctoria are contained as the at least one indican-containing component i) in an amount of 1-80 wt. %, preferably 2-50 wt. %, particularly preferably 3-25 wt. %, extremely preferably 4-15 wt. %, further extremely preferably 5-10 wt. %, further extremely preferably 6-7 wt. %, based on the weight of the agent.

Agents for non-oxidative dyeing that are preferred according to the invention are characterized in that the indican-containing component i) contains, based on its weight, 0.01-6 wt. % of indican. Preferred indican-containing components i) contain, based on their weight, 0.02-5 wt. %, preferably 0.05-4.5 wt. %, particularly preferably 0.1-4 wt. %, extremely preferably 0.2-3.9 wt. % of indican. Depending on the type of indican-containing component i), different sub-ranges of the previously mentioned broad ranges of indican quantities may be particularly preferred.

If the indican-containing component i) is dried and crushed plant parts of an indigo-producing plant, its indican content, in each case based on its weight, is preferably 1-6 wt. %, particularly preferably 2-5 wt. %, particularly preferably 3-4.5 wt. %, extremely preferably 3.5-4.1 wt. %.

If the indican-containing component i) is the solvent-containing extract from dried plant parts of an indigo-producing plant, its indican content, in each case based on its weight, is preferably 0.01-2 wt. %, particularly preferably 0.02-1 wt. %, particularly preferably 0.1-0.5 wt. %, extremely preferably 0.2-0.4 wt. %.

Dyes and dyeing methods that are preferred according to the invention are characterized in that cysteine is contained in an amount of 0.1-5.0 wt. %, preferably 0.5-3.0 wt. %, particularly preferably 0.8-2.0 wt. %, extremely preferably 1.0-1.5 wt. %, based on the weight of the ready-to-use dye or is applied to the keratin fibers.

According to the invention, the ready-to-use dye is understood to mean the agent which is obtained by mixing all the components in the compartments of the dyeing kit according to the invention and which is intended and used for immediate application to the keratin fibers. Preferably, the application of the ready-to-use dye according to the invention or preferred according to the invention to the keratin fibers takes place within 1 second to 30 minutes, particularly preferably within 10 seconds to 15 minutes, after completion of the preparation of the ready-to-use dye, i.e. immediately or without any delay after the preparation of the ready-to-use dye.

Cysteine, as a chiral amino acid, has a stereogenic center and can occur in a mirror-image form, namely in the form of L-cysteine and D-cysteine. Both L-cysteine and D-cysteine and the mixtures thereof are covered by the present invention. In the context of the present invention, both possible enantiomers can therefore equally be used as a specific compound or else mixtures thereof, and in particular as racemates. However, it is particularly advantageous according to the invention to use the naturally occurring isomer form, in this case L-cysteine.

Dyes and dyeing methods that are extremely preferred according to the invention are characterized in that L-cysteine is contained in an amount of 0.1-5.0 wt. %, preferably 0.5-3.0 wt. %, particularly preferably 0.8-2.0 wt. %, extremely preferably 1.0-1.5 wt. %, based on the weight of the ready-to-use dye or is applied to the keratin fibers.

An aqueous solution of cysteine reacts acidically. A 1 wt. % solution of L-cysteine in deionized water has a pH of 6.49, measured at 20° C. Cysteine is therefore not a basifying agent.

Further dyes and dyeing methods that are preferred according to the invention are characterized in that isatin is contained in an amount of 0.01-1.0 wt. %, preferably 0.05-0.5 wt. %, particularly preferably 0.08-0.3 wt. %, extremely preferably 0.1-0.2 wt. %, based on the weight of the ready-to-use dye, or is applied to the keratin fibers.

For the effects according to the invention, it has proven to be particularly advantageous if cysteine is contained in molar excess with respect to isatin or is applied to the keratin fibers.

Further dyes and dyeing methods that are preferred according to the invention are therefore characterized in that cysteine is contained in molar excess with respect to isatin or is applied to the keratin fibers.

Dyes and dyeing methods that are particularly preferred according to the invention are characterized in that the molar ratio of cysteine to isatin is 1.5 to 20, preferably 3 to 18, particularly preferably 5 to 15, extremely preferably 10 to 13.

The dyes according to the invention also contain water. The water is substantially used as a solvent for the enzymes, the at least one indican, cysteine and isatin. If dried and crushed plant parts of an indigo- or indirubin-producing plant which have previously been subjected to heat treatment are used as the indican-containing component i), these are preferably dispersed in water in order to extract the indican and make it accessible for the reaction to form indigo or indirubin.

If the indican-containing component i) is the solvent-containing extract from dried plant parts of an indigo- or indirubin-producing plant, it preferably contains water as the solvent.

### Water in the Ready-to-Use Dye

Ready-to-use dyes that are preferred according to the invention are characterized in that they contain water in an amount of 19.9-95.0 wt. %, preferably 30.0-90.0 wt. %, particularly preferably 50.0-88.0 wt. %, and extremely preferably 60.0-85.0 wt. %, in each case based on their weight.

### Water in the Indican-Containing Extract

If the indican-containing component i) is the solvent-containing extract from dried plant parts of an indigo-producing plant, it preferably contains water in an amount of 0.5-99.5 wt. %, more preferably 10.0-95.0 wt. %, particularly preferably 30.0-90.0 wt. %, extremely preferably 50.0-85.0 wt. %, and more extremely preferably 65-80 wt. %, in each case based on its weight.

### Water in the Indican-Containing Plant Powder Dispersion

If dried and crushed plant parts of an indigo- or indirubin-producing plant which have previously been subjected to heat treatment are used as the indican-containing component i), these are preferably dispersed in water, wherein this dispersion preferably contains water in an amount of 15.0-99.5 wt. %, more preferably 30.0-95.0 wt. %, particularly preferably 50.0-90.0 wt. %, extremely preferably 60.0-85.0 wt. %, and more extremely preferably 70-80 wt. %, in each case based on its weight.

### Water in the Indican-Containing Dry Extract Dispersion

If the dried extract from the plant parts of an indigo-producing plant is used as the indican-containing component i), this is preferably dispersed or dissolved in water, wherein this dispersion or solution preferably contains water in an amount of 15.0-99.5 wt. %, more preferably 30.0-95.0 wt. %, particularly preferably 50.0-90.0 wt. %, extremely preferably 60.0-85.0 wt. %, and more extremely preferably 70-80 wt. %, in each case based on its weight.

### Water in the Composition (E) Containing the Enzyme Mixture ii)

The enzyme mixture ii) used according to the invention comprising at least one endo-1,4-beta-glucanase (EC 3.2.1.4), at least one beta-glucosidase (EC 3.2.1.21), and at least one exo-1,4-beta-D-glucanase (EC 3.2.1.91) may preferably be dispersed or dissolved in water before it is mixed with the indican-containing component i) to form a dye according to the invention. This enzyme dispersion preferably contains water in an amount of 15.0-99.5 wt. %, more preferably 30.0-95.0 wt. %, particularly preferably 50.0-90.0 wt. %, extremely preferably 60.0-80.0 wt. %, further extremely preferably 64-75 wt. %, in each case based on its weight.

### Water as a Component of the Optional Compartments

The water content of the dye according to the invention can also be influenced by at least one further component which is contained in a compartment that is physically separate from the other components and which does not differ from the indican-containing component i) or from the enzyme mixture ii) that is used according to the invention.

The cysteine used according to the invention can be contained in at least one of the compartments selected from the indican-containing extract, the indican-containing plant powder dispersion, the composition (E) containing the enzyme mixture ii) and a further, optional compartment which, based on its weight, preferably additionally contains 0.05-99.9 wt. % water.

The isatin used according to the invention can be contained in at least one of the compartments selected from the indican-containing extract, the indican-containing plant powder dispersion, the composition (E) containing the enzyme mixture ii) and a further, optional compartment which, based on its weight, preferably additionally contains 0.05-99.9 wt. % water.

In order to save packaging material, it is preferred according to the invention that the cysteine and the isatin are contained in the compartment comprising the indican-containing component.

The dye according to the invention is further characterized in that it contains an enzyme mixture comprising
at least one endo-1,4-beta-glucanase (EC 3.2.1.4),
at least one beta-glucosidase (EC 3.2.1.21), and
at least one exo-1,4-beta-D-glucanase (EC 3.2.1.91)

Surprisingly, it was found that with an enzyme mixture used according to the invention, colorations can be achieved in which the final color result is already achieved shortly after completion of the actual dyeing process and no longer changes over time either. However, if an indican-containing component that naturally contains enzymes is used, a very unstable dyeing result is obtained, the color tone of which shifts significantly within two weeks after dyeing.

The enzymes contained in the enzyme mixture ii) used according to the invention are selected from a group of enzymes generally referred to as cellulases.

The term “cellulase” as used herein refers to enzymes that catalyze the hydrolysis of 1,4-beta-D-glucoside bonds that are present in cellulose (cellobiose) and/or lichenin and/or beta-D-glucans. Cellulases are often also able to hydrolyze the 1,4 bonds in beta-D-glucans, which also have 1,3 bonds in addition to the 1,4 bonds. Cellulases are able to break down cellulose to beta-glucose. Consequently, cellulases act in particular upon cellulose-containing or cellulose derivative-containing functional groups and catalyze their hydrolysis. The determining factor as to whether an enzyme is a cellulase in the context of the invention is its ability to hydrolyze 1,4-beta-D-glucoside bonds in cellulose.

The term “cellulase activity” is defined here as an enzyme that catalyzes the hydrolysis of 1,4-beta-D-glucoside bonds in beta-1,4-glucan (cellulose). Cellulose activity is measured using a standard method, e.g., as follows: Cellulases release glucose from CMC (carboxymethylcellulose). The samples are incubated under defined reaction conditions (100 mM sodium phosphate buffer pH 7.5, 40° C., 15 min) with a substrate (1.25 wt. % CMC). The reaction with p-hydroxybenzoic acid hydrazide (PAHBAH) in the presence of bismuth produces a yellow dye that can be determined photometrically at 410 nm. The prerequisite is an alkaline pH during the color reaction. The amount of sugar released corresponding to the coloration is a measure of enzyme activity (Lever, Anal. Biochem., 1972, 47 & 1977, 81).

Cellulases can be divided into three categories:
1. Endoglucanase (EC 3.2.1.4), also called endo-1,4-beta-glucanase, beta-1,4-glucanase, avicelase, beta-1,4-endoglucanhydrolase, endo-1,4-beta-D-glucanohydrolase, carboxymethylcellulase, or celludextrinase;
2. Cellulose-1,4-beta-cellobiosidase (non-reducing end) (EC 3.2.1.91), also called exoglucanase, 1,4-beta-cellobiohydrolase, 4-beta-D-glucan cellobiohydrolase (non-reducing end), avicelase, exo-1,4-beta-D-glucanase, or exocellobiohydrolase, releases cellobiose from the non-reducing ends of the β-D-glucan chains by hydrolysis of the (1->4)-beta-D-glucosidic bonds in cellulose and cellotetraose;
3. Beta-glucosidase (EC 3.2.1.21), also called cellobiase, beta-D-glucoside glucohydrolase, amygdalase, or gentobiase, hydrolyzes terminal, non-reducing beta-D-glucosyl groups to release beta-D-glucose.

Cellulases that are preferably used according to the invention do not originate from plants, but are of bacterial or fungal origin. For the production of cellulases which are particularly preferably used according to the invention, fungi are used which are selected from Aspergillus spp., in particular Aspergillus niger, Aspergillus niger SOI017, Aspergillus niger AS 3.4309, Aspergillus niger HDF 05, Aspergillus oryzae, Aspergillus strain SA 58, Aspergillus terreus EMOO 6-4, and mixtures of Aspergillus niger and Aspergillus oryzae, and also selected from Trichoderma reesei. Chemically modified or protein-engineered mutants are included.

Examples of cellulases with endo-1,4-glucanase activity (EC 3.2.1.4) are described in WO 2002/099091, for example those having a sequence of at least 97% identity to the amino acid sequence of positions 1 to 773 of SEQ ID NO:2 of WO 2002/099091.

A preferred embodiment of the invention is characterized in that the agent for non-oxidative dyeing contains an enzyme mixture comprising at least one endo-1,4-beta-glucanase (EC 3.2.1.4) in a total amount of 20-35 wt. %, at least one beta-glucosidase (EC 3.2.1.21) in a total amount of 15-25 wt. %, and at least one exo-1,4-beta-D-glucanase (EC 3.2.1.91) in a total amount of 25-35 wt. %, wherein the stated amounts relate to the total weight of the enzyme mixture ii).

In a further preferred embodiment of the invention, in addition to the at least one endo-1,4-beta-glucanase (EC 3.2.1.4), the at least one beta-glucosidase (EC 3.2.1.21), and the at least one exo-1,4-beta-D-glucanase (EC 3.2.1.91), at least one further enzyme with cellulase activity is additionally contained, which is selected from exo-1,4-beta-glucosidase (EC 3.2.1.74), cellulose-1,4-beta-cellobiosidase (EC 3.2.1.176), and oligoxyloglucan reducing-end-specific cellobiohydrolase (EC 3.2.1.150), and mixtures of these enzymes.

A further particularly preferred embodiment of the invention is characterized in that the agent for non-oxidative dyeing contains an enzyme mixture comprising at least one endo-1,4-beta-glucanase (EC 3.2.1.4) in a total amount of 20-35 wt. %, at least one beta-glucosidase (EC 3.2.1.21) in a total amount of 15-25 wt. %, and at least one exo-1,4-beta-D-glucanase (EC 3.2.1.91) in a total amount of 25-35 wt. %, and at least one further enzyme with cellulase activity selected from exo-1,4-beta-glucosidase (EC 3.2.1.74), cellulose-1,4-beta-cellobiosidase (EC 3.2.1.176), and oligoxyloglucan reducing-end-specific cellobiohydrolase (EC 3.2.1.150) and mixtures of these enzymes in a total amount which, together with the amounts of the other enzymes, makes up 100 wt. %, the stated amounts referring to the total weight of the enzyme mixture ii).

A further particularly preferred embodiment of the invention is characterized in that the agent for non-oxidative dyeing contains, based on its weight, an enzyme mixture with cellulase activity, the at least one endo-1,4-beta-glucanase (EC 3.2.1.4), at least one beta-glucosidase (EC 3.2.1.21), and at least one exo-1,4-beta-D-glucanase (EC 3.2.1.91) in a total amount of 0.0001-1 wt. %, preferably 0.001-0.1 wt. %, more preferably 0.002-0.05 wt. %, particularly preferably 0.003-0.04 wt. %, extremely preferably 0.01-0.03 wt. %, further extremely preferably 0.015-0.02 wt. %.

The precursor of the dyes indigo and indirubin is indican. Indican, which occurs naturally in plants, is a glycoside, more precisely a glucoside, which is split into indoxyl and the glycoside functional group before the indican is converted into the dye indigo or indirubin. This cleavage process preferably occurs enzymatically. The cleavage process should occur in situ, i.e. just before application to the keratin fibers to be dyed.

The above-discussed agent for non-oxidatively dyeing keratin fibers is therefore preferably sold commercially as a kit consisting of at least 2 components (Ind) and (E), which are physically separated from one another.

The present invention therefore also relates to a kit for non-oxidatively dyeing keratin fibers, in particular human hair, with natural dyes, which kit comprises the following compartments that are physically separated from one another:
i) a composition (Ind) containing at least one indican-containing component which has been pretreated at a temperature in the range of 60° C. to 200° C., and
ii) an aqueous composition (E) containing an enzyme mixture comprising at least one endo-1,4-beta-glucanase (EC 3.2.1.4), at least one beta-glucosidase (EC 3.2.1.21), and at least one exo-1,4-beta-D-glucanase (EC 3.2.1.91),
iii) optionally at least one further compartment,
   characterized in that at least one of the compartments contains cysteine and at least one of the compartments contains isatin.

The enzyme mixture ii) used according to the invention comprising at least one endo-1,4-beta-glucanase (EC 3.2.1.4), at least one beta-glucosidase (EC 3.2.1.21), and at least one exo-1,4-beta-D-glucanase (EC 3.2.1.91), may preferably be dispersed or dissolved in water before it is mixed with the indican-containing component i), the cysteine and the isatin to form a ready-to-use dye according to the invention. This enzyme dispersion, which represents component (E) of the kit according to the invention, preferably contains, in each case based on its weight, water in an amount of 15.0-99.5 wt. %, more preferably 30.0-95.0 wt. %, particularly preferably 50.0-90.0 wt. %, extremely preferably 60.0-80.0 wt. %, more extremely preferably 64-75 wt. %.

The enzyme active substance content in the aqueous composition (E) is preferably, in each case based on its weight, 1-70 wt. %, particularly preferably 5-50 wt. %, further particularly preferably 10-35 wt. %, extremely preferably 15-25 wt. %, further extremely preferably 18-22 wt. %.

Further dyes that are preferred according to the invention are characterized in that saponins are contained in a total amount of from zero to less than 0.01 wt. %, preferably from zero to 0.005 wt. %, particularly preferably from zero to 0.001 wt. %, in each case based on the weight of the ready-to-use dye.

The statements made about the dyes according to the invention apply, mutatis mutandis, to other preferred embodiments of the dyeing kit according to the invention.

The present invention also relates to a method for non-oxidatively dyeing keratin fibers, in particular human hair, which comprises the following method steps in the order given:
a) providing a composition (Ind) which contains at least one indican-containing component which has been pretreated at a temperature in the range of 60° C. to 200° C., and providing an aqueous composition (E) which contains an enzyme mixture comprising at least one endo-1,4-beta-glucanase (EC 3.2.1.4), at least one beta-glucosidase (EC 3.2.1.21), and at least one exo-1,4-beta-D-glucanase (EC 3.2.1.91), and optionally providing at least one further component in a physically separate compartment,
b) wherein at least one of the compositions (Ind) or (E) or at least one optional component contains cysteine and at least one of the compositions (Ind) or (E) or at least one optional component contains isatin,
c) mixing the compositions mentioned under a) above with one another to produce a ready-to-use dye,
d) applying the ready-to-use dye obtained in step b) to the keratin fibers,
e) allowing to act for a time of from 30 seconds to 60 minutes, preferably 5 to 45 minutes, particularly preferably 15 to 30 minutes,
f) rinsing the keratin fibers with water,
g) optionally drying the keratin fibers.

The ready-to-use dye obtained in step b) is applied to the keratin fibers. Preferably, the ready-to-use dye obtained in step b) is applied to the keratin fibers within 1 second to 30 minutes, particularly preferably within 10 seconds to 15 minutes after completion of method step b), i.e. immediately after the production of the ready-to-use dye.

Surprisingly, it was found that the development over time of the dyeing result on the keratin fibers can be further accelerated if the ready-to-use dye according to the invention is left to act under exposure to heat, for example by means of a heat lamp or a drying hood or a hair dryer.

A particularly preferred embodiment of the dyeing method according to the invention is therefore characterized in that the ready-to-use dye obtained in step b) is left to act under exposure to heat, preferably at a temperature of 25-60° C., particularly preferably at a temperature of 30-50° C., extremely preferably at a temperature of 35-40° C.

In conventional dyes and dyeing methods with natural dyes, compounds, in particular salts, of metals other than alkali metals are often used to improve the adhesion of the natural dye to the keratin fibers. Such compounds are also called “mordants”. For example, US20040055096A1 discloses pretreating or post-treating the keratin fibers with metals or metal salts selected from aluminum, iron, zinc, nickel, calcium and magnesium. Other mordants are transition metals and lanthanides as well as the salts and complex compounds thereof.

The content of metal ions other than alkali metal ions that is usually present in the water used, e.g. in tap water or city water, is not taken into account here. Tap water can contain an average of 2 mg of copper ions per liter, i.e. approximately 0.0002 wt. % of copper ions. Tap water contains about 20-120 mg calcium ions per liter, i.e. about 0.002-0.012 wt. % calcium ions. Tap water contains about 2-25 mg magnesium ions per liter, i.e. about 0.0002-0.0025 wt. % magnesium ions. At such low concentrations, no noticeable mordant effect occurs. For the purposes of the present invention, the agents according to the invention are “free of mordants” if the mordant(s) is/are contained in maximum concentrations that are naturally the standard concentration for the ingredients used, such as water, enzymes and plant parts.

The dyes, dyeing kits and dyeing methods according to the invention can dispense with the use of such mordants.

Preferred dyes and dyeing methods according to the invention are therefore characterized in that they are free of mordants. In order to preserve the hair-protecting potential of natural dyes, methods that are preferred according to the invention are limited to methods in which the keratin fibers have not been treated with a mordant within a period of up to 7 days prior to, and up to 7 days after, the application of a dye according to the invention.

Further preferred dyes, dyeing kits and dyeing methods according to the invention are characterized in that they are free of hydrogen peroxide. Further preferred dyes, dyeing kits and dyeing methods according to the invention are characterized in that they are free of mordants and hydrogen peroxide.

The statements made in relation to the dyes and dyeing kits according to the invention apply, mutatis mutandis, to further preferred embodiments of the dyeing method according to the invention.

The keratin fibers to be dyed, to which the ready-to-use dye prepared in step b) by mixing the compositions provided in step a) is applied in method step c), are preferably dry.

In order to preserve the hair-protecting potential of natural dyes, the claimed method is preferably limited to those methods in which the keratin fibers have not been treated with an oxidizing agent within a period of up to 7 days prior to the application of the dye according to the invention.

The oxidizing agents which are usually used in hair cosmetics but which, according to the invention, are not intended to be used to treat hair, not even as a pre-treatment, include hydrogen peroxide, persulfates, perbromates, percarbonates, perborates and percarbamides. The oxygen contained in the ambient air does not constitute an oxidizing agent within the context of the invention.

In order to preserve the hair-protecting potential of natural dyes, the methods that are preferred according to the invention are limited to methods in which the keratin fibers have not been treated with a keratin-reducing compound within a period of up to 7 days prior to the application of a dye according to the invention.

Preferably, the keratin fibers are dried after rinsing out the ready-to-use dye. Drying can take place without the active supply of heat. However, drying can also be carried out with a supply of heat at a temperature of 25-120° C., particularly preferably at a temperature of 30-80° C., extremely preferably at a temperature of 35-60° C. Heat is preferably supplied by a heat lamp, a drying rod, a drying hood, a straightening iron, or a hair dryer.

A further feature of the dyeing method according to the invention is that the ready-to-use dye is left to act on the keratin fibers after application thereto for 30 seconds to 60 minutes, preferably 5 to 45 minutes, particularly preferably 20 to 35 minutes, extremely preferably 25 to 30 minutes.

After the development time of the dye has elapsed, the keratin fibers are rinsed with water to wash out the dye.

The keratin fibers can optionally be dried after this rinsing step. The drying can take place using an absorbent cloth, such as a towel. The towel-dried keratin fibers can optionally also be partially or completely dried with a hairdryer or other heat source. It is also possible to let the keratin fibers air dry.

Further dyeing methods that are preferred according to the invention are characterized in that no oxidation dye precursors are used therein. Typical oxidation dye precursors are p-aminophenol, 4-amino-3-methylphenol, 4-amino-2-aminomethylphenol, 4-amino-2-(1,2-dihydroxyethyl) phenol, 4-amino-2-(diethylaminomethyl) phenol, 2-(2,5-diaminophenyl) ethanol, 2-(1,2-dihydroxyethyl)-p-phenylenediamine, N,N-bis(2-hydroxyethyl)-p-phenylenediamine, N-(4-amino-3-methylphenyl)-N-[3-(1H-imidazol-1-yl) propyl]amine, N,N′-bis(2-hydroxyethyl)-N,N′-bis(4-aminophenyl)-1,3-diaminopropan-2-ol, bis(2-hydroxy-5-aminophenyl) methane, 1,3-bis(2,5-diaminophenoxy) propan-2-ol, N,N′-bis(4-aminophenyl)-1,4-diazacycloheptane, 1,10-bis(2,5-diaminophenyl)-1,4,7,10-tetraoxadecane, 2,4,5,6-tetraaminopyrimidine, 4-hydroxy-2,5,6-triaminopyrimidine, 2-hydroxy-4,5,6-triaminopyrimidine, 2,3-diamino-6,7-dihydro-1H,5H-pyrazolo-[1,2-a]-pyrazol-1-one, 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)amino-2-methylphenol, 2,4-dichloro-3-aminophenol, 2-aminophenol, 3-phenylenediamine, 2-(2,4-diaminophenoxy) ethanol, 1,3-bis(2,4-diaminophenoxy) propane, 1-methoxy-2-amino-4-(2-hydroxyethylamino)benzene, 1,3-bis(2,4-diaminophenyl) propane, 2,6-bis(2′-hydroxyethylamino)-1-methylbenzene, 2-({3-[(2-hydroxyethyl)amino]-4-methoxy-5-methylphenyl}amino) ethanol, 2-({3-[(2-hydroxyethyl)amino]-2-methoxy-5-methylphenyl}amino) ethanol, 2-({3-[(2-hydroxyethyl)amino]-4,5-dimethylphenyl}amino) ethanol, 2-[3-morpholin-4-ylphenyl)amino]ethanol, 3-amino-4-(2-methoxyethoxy)-5-methylphenylamine, 1-amino-3-bis(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 2-(1,3-benzodioxol-5-ylamino) ethanol, (1-beta-hydroxyethyl-3,4-methylenedioxyaniline), 4-hydroxyindole, 6-hydroxyindole, 7-hydroxyindole, 4-hydroxyindoline, 6-hydroxyindoline and 7-hydroxyindoline.

The compositions (Ind) and (E) used according to the invention and preferably used according to the invention and the at least one optional component in the separate compartment can—independently of one another—optionally contain further additives which can optimize their application properties. Preferred additives are in particular thickeners which ensure that the ready-to-use dye according to the invention or preferred according to the invention, prepared from the compositions (Ind) and (E) and optionally further optional components in separate compartments, remains on the keratin fibers better during application.

Compositions (Ind) and (E) that are particularly preferably used according to the invention contain—independently of one another—at least one or more hydrophilic thickeners preferably selected from polysaccharides other than cellulose and can be chemically and/or physically modified. Compounds from the group of polysaccharides are particularly preferred according to the invention as hydrophilic thickeners, since the basic structure of polysaccharides are of natural origin and biodegradable. Preferred hydrophilic polysaccharide thickeners are selected from xanthan gum, alginic acids (and their corresponding physiologically acceptable salts, the alginates), agar-agar (with the polysaccharide agarose that is present as the main component of agar-agar), starch fractions and starch derivatives, such as amylose, amylopectin and dextrins, gum karaya, locust bean gum, gum arabic, pectins, dextrans and guar gum, and mixtures thereof.

In embodiments that are preferred with regard to reliable viscosity adjustment and residue-free application to keratin fibers and the scalp, xanthan gum is contained as the hydrophilic thickener.

Compositions (Ind) and (E) that are particularly preferably used according to the invention independently contain, in each case based on the weight, at least one hydrophilic thickener in a total amount of 0.1 to 5 wt. %, preferably of 0.5 to 4 wt. %, more preferably of 1 to 3.5 wt. %, and very particularly preferably of 1.2 to 2 wt. %.

In a further preferred embodiment of the present invention, the compositions (Ind) and (E) used according to the invention independently contain, in each case based on their weight, 0.1 to 3 wt. %, preferably 0.5 to 2.5 wt. %, more preferably 1.2 to 2.0 wt. %, of xanthan gum.

Compositions (Ind) and (E) that are particularly preferably used according to the invention contain, independently of one another, at least one organic solvent that has a phenyl group in the molecule. Preferably, this solvent is selected from phenoxyethanol, benzyl alcohol, and mixtures thereof. Surprisingly, it was found that such aromatic solvents can have a positive effect on the dyeing results of the dyeing method according to the invention; this was observed in particular for aromatic solvents.

In a further preferred embodiment of the present invention, the compositions (Ind) and (E) that are used according to the invention independently contain, in each case based on their weight, 0.1 to 3 wt. %, preferably 0.5 to 2.5 wt. %, more preferably 0.8 to 1.0 wt. %, of at least one organic solvent that has a phenyl group in the molecule. In a further preferred embodiment of the present invention, the compositions (Ind) and (E) that are used according to the invention independently contain, in each case based on their weight, 0.1 to 3 wt. %, preferably 0.5 to 2.5 wt. %, more preferably 0.8 to 1.0 wt. %, of at least one organic solvent selected from phenoxyethanol, benzyl alcohol and mixtures thereof.

Compositions (Ind) and (E) that are particularly preferably used according to the invention contain, independently of one another, at least one aliphatic solvent selected from C₁-C₄ alkanols and C₂-C₄ polyols, in particular selected from ethanol, isopropanol, n-propanol, ethylene glycol, 1,2-propanediol, glycerin and 1,3-butylene glycol, and mixtures of these solvents, but only in a total amount of 0.01-8 wt. %, preferably 0.1-6 wt. %, particularly preferably 0.5-4 wt. %, in each case based on the weight of the compositions (Ind) or (E).

Other compositions (Ind) and (E) that are particularly preferably used according to the invention do not contain, independently of one another, an aliphatic solvent selected from C₁-C₄ alkanols and C₂-C₄ polyols.

The statements made above with regard to the compositions (Ind) and (E) also apply, mutatis mutandis, to the at least one optional component in the at least one optional physically separate compartment.

Powders from the leaves of Indigofera tinctoria which are preferably used according to the invention react approximately neutrally in aqueous dispersion, with slight deviations between a slightly acidic and a slightly alkaline pH range. A 5 wt. % dispersion of ground leaves of Indigofera tinctoria in deionized water has a pH of 6.6 to 7.4, preferably 6.8 to 7.2, particularly preferably 6.9 to 7.1, in each case measured at 20° C.

Ready-to-use dyes that are preferred according to the invention have a pH of 2.0 to less than 10.0, preferably 4.0 to 9.0, particularly preferably 5.0 to less than 8.0, extremely preferably 6.6 to less than 7.5, in each case measured at 20° C.

In a preferred embodiment of the dyeing method according to the invention, the desired pH is set using an acid or a base. Preferred acids are selected from citric acid, lactic acid, gluconic acid, acetic acid, propionic acid, oxalic acid, malonic acid, succinic acid, glutaric acid, galactaric acid (mucic acid), tartaric acid, malic acid, sulfuric acid and phosphoric acid as well as mixtures of these acids. Preferred bases are sodium hydroxide, potassium hydroxide, arginine, lysine, monoethanolamine, triethanolamine, 2-amino-2-methylpropan-1-ol and mixtures of these bases.

In a further preferred embodiment of the dyeing method according to the invention, the desired pH is set using a buffer system selected from a mixture of a medium-strength or weak acid with its conjugated or corresponding base (or the corresponding salt) and a mixture of a medium-strength or weak base with its conjugated or corresponding acid.

Suitable corresponding acid-base pairs that are preferred according to the invention are those which stabilize the compositions used according to the invention in the pH range of 2.0 to less than 10.0, preferably 4.0 to 9.0, particularly preferably 5.0 to less than 8.0, extremely preferably 6.6 to less than 7.5, in each case measured at 20° C.

Particularly preferred buffer systems according to the invention are selected from
ammonia-ammonium salt mixtures, wherein the ammonium salt is preferably selected from ammonium chloride, ammonium bromide, ammonium hydrogen sulfate, ammonium sulfate, ammonium dihydrogen phosphate, diammonium hydrogen phosphate, ammonium phosphate, ammonium bicarbonate, ammonium carbonate, ammonium nitrate, ammonium acetate, ammonium glycolate, ammonium gluconate, ammonium tartrate, ammonium lactate, and mixtures of these ammonium salts, particularly preferably selected from ammonium chloride, ammonium hydrogen sulfate, ammonium sulfate, ammonium dihydrogen phosphate, diammonium hydrogen phosphate, ammonium phosphate, ammonium bicarbonate and ammonium carbonate, extremely preferably selected from ammonium chloride,
Mixtures of hydrogen phosphate and dihydrogen phosphate, in particular the alkali metal salts of hydrogen phosphate and dihydrogen phosphate, particularly preferably the sodium salts and/or the potassium salts of hydrogen phosphate and dihydrogen phosphate,
Mixtures of alkali metal hydrogen carbonate with alkali metal carbonate, in particular mixtures of sodium hydrogen carbonate or potassium hydrogen carbonate with sodium carbonate or potassium carbonate,
Mixtures of citric acid and its salts, in particular alkali metal citrates, in particular sodium salts, in particular trisodium citrate,
Mixtures of tartaric acid and its salts, in particular alkali metal tartrates, in particular potassium salts, in particular potassium hydrogen tartrate,
Mixtures of phthalic acid and its salts, in particular potassium salts, in particular potassium hydrogen phthalate,
Mixtures of lactic acid and its salts, in particular lactic acid-sodium lactate mixtures,
Mixtures of gluconic acid and its salts, in particular gluconic acid-sodium gluconate mixtures,
Mixtures of succinic acid and its salts, in particular sodium salts, in particular sodium hydrogen succinate and disodium succinate, and
Mixtures of malic acid and its salts, in particular sodium salts, in particular sodium hydrogen malate and disodium malate, as well as
Ammonia-ammonium salt mixtures, wherein the ammonium salt is preferably selected from ammonium chloride, ammonium bromide, ammonium hydrogen sulfate, ammonium sulfate, ammonium dihydrogen phosphate, diammonium hydrogen phosphate, ammonium phosphate, ammonium bicarbonate, ammonium carbonate, ammonium nitrate, ammonium acetate, ammonium glycolate, ammonium gluconate, ammonium tartrate, ammonium lactate, and mixtures of these ammonium salts, particularly preferably selected from ammonium chloride, ammonium hydrogen sulfate, ammonium sulfate, ammonium dihydrogen phosphate, diammonium hydrogen phosphate, ammonium phosphate, ammonium bicarbonate and ammonium carbonate, extremely preferably selected from ammonium chloride.

Other buffer systems, e.g. acetic acid/sodium acetate, are in principle also suitable according to the invention. However, due to the vinegary smell, such a buffer is not acceptable for the preparation of a cosmetic market product.

However, in principle, other buffer systems are also suitable.

Those skilled in the art can find out the appropriate weights of buffer salts for setting the desired pH from relevant manuals.

### EMBODIMENTS

The exemplary embodiments presented below are intended to explain the subject matter of the invention in more detail, without limiting it thereto.

The dyeing processes for this invention were carried out on buffalo belly hair strands (tied as a brush body, approx. 8 cm free hair).

### Preparation of the Indican-Containing Component i) (Composition (Ind))

95 ml of demineralized water were heated to approximately 95° C. Subsequently, 5 grams of ground leaves of Indigofera tinctoria L.* were added (5 wt. % of the total dispersion) and stirred for 5 minutes at approximately 95° C.

* The following raw materials were used:
“C30 Indigo Leaves Powder” (Indigofera tinctoria L.); powdered leaves of Indigofera tinctoria pH of the 5-wt. % dispersion in water: 6.8 to 7.2 (20° C.)
or
“Indigo Powder” (Indigofera tinctoria L.); powdered leaves of Indigofera tinctoria
pH of the 5-wt. % dispersion in water: 6.8 to 7.2 (20° C.)

The dispersion was then cooled to room temperature (20° C.) and filtered (filter: Whatman 10340810, viscose, pore size 100 μL, approx. 5 to 10 min, vacuum pump at approx. 500 mbar).

The filtrate was a brown, opaque solution without precipitate, with a pH of approximately 7.7 (20° C.). This indican-containing component i) will hereinafter be referred to as “composition (Ind)”.

Treatment of hair strands with the composition (Ind) does not dye the hair strands under standard dyeing conditions (50 ml of composition (Ind) per 1 g of hair strand, dye for 30 minutes at room temperature (20° C.) while stirring, rinse for 30 seconds under running deionized water, comb 20 times at 20° C., dry with hairdryer, distance: d=10 cm, temperature: 80±5° C., comb 20 times).

### Dyeing Process According to the Invention

The aqueous composition (E) according to the invention, which contains an enzyme mixture ii) according to the invention that comprises at least one endo-1,4-beta-glucanase (EC 3.2.1.4), at least one beta-glucosidase (EC 3.2.1.21), and at least one exo-1,4-beta-D-glucanase (EC 3.2.1.91), was obtained from Novozymes. This aqueous composition (E) will hereinafter be referred to as a “cellulase blend”.

The enzyme mixture ii) in the “cellulase blend” from Novozymes contained at least one endo-1,4-beta-glucanase (EC 3.2.1.4) in a total amount of 20-35 wt. %, at least one beta-glucosidase (EC 3.2.1.21) in a total amount of 15-25 wt. %, and at least one exo-1,4-beta-D-glucanase (EC 3.2.1.91) in a total amount of 25-35 wt. %, and at least one other enzyme with cellulase activity in a total amount which, together with the amounts of the other enzymes, makes up 100 wt. %, the amounts stated referring to the total weight of the enzyme mixture ii).

The enzyme active substance content in the raw material “cellulase blend” by Novozymes that is used, which was present as an aqueous composition, was 190 μg/μl, i.e. 19 wt. %. Furthermore, the “cellulase blend” contained, based upon its weight, 66 wt. % water and 15 wt. % of a sucrose/D-glucose mixture.

To prepare the dyeing composition according to the invention, 0.5 g of L-cysteine and 0.05 g of isatin were added to 49.4 ml of the composition (Ind) according to the invention and dissolved. While stirring, this dispersion was heated to about 37° C. on a stir plate. When this temperature was reached, 0.05 ml of the “cellulase blend” from Novozymes described above was added. The resulting dyeing composition according to the invention had a total enzyme content of 0.019 wt. %, based on its weight. This ready-to-use dye had a pH of 7.0 measured at 20° C.

A strand of buffalo belly hair (tied to make a round shape, about 8 cm of free hair) was placed in this ready-to-use dye, the temperature of which was 37° C., for 30 minutes and stirred at 37° C.

The liquor ratio, i.e. the weight of dye per gram of hair, was 50 g of dye per 1 g of hair (50:1).

After dyeing, the hair strands were rinsed under running deionized water for 30 seconds while being combed 20 times (20° C.).

The hair strands were then dried with a commercially available hair dryer at a defined distance (d=10 cm) and a defined temperature (T=80±5° C.) while combing them 20 times.

Determination of dyeing results using spectrophotometric measurements

All colorimetric measurements were carried out using the Spectraflash SF 600 colorimetric device from Datacolor.

The color difference, also known as dE or ΔE, can be easily determined colorimetrically using a colorimeter that measures colors in the L*, a*, b* color space—or example, a Datacolor colorimeter, type Spectraflash SF 600.

The L*, a*, b* color space refers to the CIELAB color space. The L value represents the brightness of the color (black-white axis); the greater the L value, the brighter the color. The a value represents the red-green axis of the system; the greater this value, the more the color is shifted towards red. The b value represents the yellow-blue axis of the system; the greater this value, the more the color is shifted towards yellow.

The color shift ΔE, i.e., the color difference between two (hair) colors, for each of which an L*, a*, b* value combination was determined, is calculated according to the following formula:

$$Δ E = {( Δ {L}^{2} + Δ {a}^{2} + Δ {b}^{2} )}^{0.5}$$

The greater the value for ΔE, the more pronounced the color difference.

For the spectrophotometer measurements, a D65 illuminant and a diffuse/8° optical configuration were used. The spectral reflectance data for each sample from 380 nm to 700 nm were converted to colorimetric data using DCI Color software. Reflection measurements were taken for each hair sample, with the average of 4 measurements being recorded.

The color difference (ΔE) between undyed strand and dyed strand was calculated according to the following formula:

$$Δ E = \sqrt{{( Lv - Ln )}^{2} + {( av - an )}^{2} + {( bv - bn )}^{2}}$$

where
Lv, av, bv: colorimetric values for colored strand
Ln, an, bn: colorimetric values for uncolored strand

In order to also track the development of the final color over time, measurements were taken immediately after dyeing on day 0 and 14 days after dyeing and compared with one another.

On day 0 immediately after dyeing, the strands had an intense magenta color. The change in the color tone of this strand within the two weeks after dyeing became even more uniform overall (ΔE of 11.6) (see Table 1).

The differences between the strands dyed according to the invention and the strands analogously dyed with the composition (Ind) and cellulase blend but without cysteine and isatin immediately after dyeing (day 0) and 14 days after dyeing are shown in Table 1 (averages).

TABLE 1
Comparison of dyeing with heat-treated composition (Ind) + reactivation by adding
cellulase, with and without cysteine-isatin combination 0 days and 14 days after dyeing

| Batch Name | CIE L sample | CIE a sample | CIE b sample | Color name |
|---|---|---|---|---|
| Day 0 | | | | |
| Heat-deactivated indigo | 53.4 | −4.6 | −4.1 | gray-blue |
| extract with enzymatic | | | | |
| reactivation, without | | | | |
| cysteine, without isatin | | | | |
| (comparison, average) | | | | |
| Heat-deactivated indigo | 40.2 | 25.3 | 0.9 | frenzied red (7421 |
| extract with enzymatic | | | | U, Pantone U) |
| reactivation, cysteine and | | | | |
| isatin (according to the | | | | |
| invention, average) | | | | |

Color difference: ΔE = 33.1

| Day 14 | | | | |
|---|---|---|---|---|
| Heat-deactivated indigo | 52.4 | −4.5 | −5.4 | ocean-gray |
| extract with enzymatic | | | | |
| reactivation, without | | | | |
| cysteine, without isatin | | | | |
| (comparison, average) | | | | |
| Heat-deactivated indigo | 40.2 | 25.3 | 0.9 | frenzied red (7421 |
| extract with enzymatic | | | | U, Pantone U) |
| reactivation, cysteine and | | | | |
| isatin (according to the | | | | |
| invention, average) | | | | |

Difference between the control and extract strand: ΔE = 32.9

The L a b values were converted into Pantone values and color names using the website https://www.e-paint.co.uk/convert-lab.asp.

The addition of cysteine and isatin to the indican-containing solution activated using cellulase results in an intense magenta tone (frenzied red (7421 U, Pantone U)) visible on the hair fiber after dyeing, rather than a blue-turquoise/blue-gray tone (see Table 1). The change in the color tone of this strand in the two weeks after coloring becomes even more uniform overall, but is negligible (ΔE of approx. 1 to 3).

In the dyeing process according to the invention that is described above, in contrast to the comparative dyeing process without cysteine and without isatin, the magenta tone can be observed on the hair strand immediately after treatment.

### Influence of the Dyeing Temperature

The same dyeing process, but carried out at 20° C., shows a pink tinge immediately after treatment. This could not be quantified due to the moisture that remained. However, an irregular, stronger red-magenta tone develops during subsequent blow-drying (temperature at a distance of 5 cm from the hairdryer: 96° C., at a distance of 15 cm from the hairdryer: 79° C.). The color difference between dyeing at 37° C. followed by blow-drying and dyeing at 20° C. followed by blow-drying was ΔE=28.0.

The differences after the dyeing process between the control strand and the strands where cysteine/isatin were added immediately after dyeing at 20° C. (day 0) are shown below (averages).

TABLE 2
L*, a*, b*, ΔE values of indican/cellulase dyes with cysteine/isatin
and indican/cellulase alone, dyeing at 20° C. and blow-drying

| Batch Name | CIE L sample | CIE a sample | CIE b sample | Color name |
|---|---|---|---|---|
| Day 0 | | | | |
| Indican/cellulase with | 59.7 | 11.4 | 15.5 | Cinnamon, caramel |
| cysteine/isatin (according | | | | brown, Renwick |
| to the invention, average) | | | | rose-beige (4655 U |
| | | | | (Pantone U)) |

Difference between the comparison and invention (dyeing at 20° C.): ΔE = 28.0

### Influence of the pH of the Dye on the Color Result

The following shows how the pH of the ready-to-use dye affects the color result.

The dyes are prepared and the strands are dyed according to the process described above. In the ready-to-use dye according to the invention consisting of indican solution, cellulase blend, 1 wt. % cysteine and 0.1 wt. % isatin in water, a pH of 7.0 is spontaneously established. The pH of other ready-to-use dyes according to the invention was set to 5, 8 and 10 using sodium hydroxide solution or hydrochloric acid, each measured at 20° C. The dyeing processes were carried out at 37° C. After dyeing, all strands were washed and dried according to the usual protocol (see above). The color results were compared with those obtained at the “spontaneous” pH of 7.0. The dye dispersion having a pH of 10 is not part of the invention (V-1).

TABLE 3
L*, a*, b*, ΔE values and color names of indican/cellulase dyes with cysteine
and isatin at 37° C. and different pHs immediately after dyeing (day 0)
Indican/cellulase with cysteine + isatin at 37° C., day 0

| pH spontaneous (7.0) | | | pH 5 (A-2) | | | pH 8 (A-3) | | | pH 10 (V-1) | | |
|---|---|---|---|---|---|---|---|---|---|---|---|
| 40.2 | 25.3 | 0.9 | 36.4 | 28.8 | −2.8 | 53.3 | 16.0 | 9.3 | 65.5 | 5.5 | 21.1 |

| frenzied red (7421 U, | Wild plum, magenta | Brown, ashes of | Latte, toasted sesame |
|---|---|---|---|
| Pantone U) | | roses (7525 U | (465 U (Pantone U)) |
| | | (Pantone U) | |
| difference with | ΔE = 6.4 | ΔE = 18.2 | ΔE = 37.9 |
| respect to | | | |
| pHₛₚₒₙₜₐₙₑₒᵤₛ: | | | |

It can be observed that the color intensity and magenta tone increase at pH 5. In a more alkaline environment, the a* value, which represents the intensity of the red color, decreases sharply. At pH 10 (not according to the invention) the desired color is not visible on the hair fiber.

The data presented led to the conclusion that the dye according to the invention produces the darkest and most intense and reddest result at pH 5. The dye in the slightly alkaline range (pH 8) comes the color result with the spontaneous pH (7.4) shows a weaker pink tone (ΔE=18.2). At a pH of 10 of the dye, no color result is achieved that is similar to the pH of 7.0 (ΔE=37.9) that is spontaneously established.

By initially deactivating the enzyme activity present in indican-containing, indigo- or indirubin-producing plant parts and later adding a chosen enzyme mixture with cellulase activity shortly before application in combination with cysteine and isatin, the dyeing performance of the composition (Ind), which initially had no dyeing ability, can be restored. Conversely, dyeing occurs immediately, which does not noticeably change within 14 days.

### Influence of the Reducing Agent

The following shows how replacing cysteine with another reducing agent affects the color result.

The dyes were prepared and the strands were dyed using the method described above. In addition to the ready-to-use dye according to the invention consisting of an indican-containing composition (Ind), cellulase blend, 1 wt. % cysteine and 0.1 wt. % isatin in water, a comparative dye (V-2) consisting of an indican-containing composition (Ind), cellulase blend, 1 wt. % ascorbic acid and 0.1 wt. % isatin in water was prepared. One batch was used to dye hair strands at 20° C. and another batch at 37° C. Both strands were then washed and dried according to the usual protocol (see above). The pH of the dispersion having the indican-containing composition (Ind), cellulase blend, ascorbic acid and isatin was spontaneously, i.e. without addition of further pH adjusters, in the range from 4.3-4.7 (measured at 20° C.). The color results obtained are summarized in Table 4 and Table 5.

TABLE 4
Comparison between treatment according to the invention with indican/cellulase
and cysteine and isatin at 37° C. and non-inventive treatment
with indican/cellulase and ascorbic acid and isatin at 37° C.
immediately after rinsing and blow-drying (L*, a*, b* values are shown)

| Color result according to the invention (day 0) | | | Non-inventive color result (day 0) | | |
|---|---|---|---|---|---|
| 40.2 | 25.3 | 0.9 | 44.9 | 19.7 | 13.2 |

| | |
|---|---|
| frenzied red (7421 U, Pantone U) | Canyon clay, red-brown (7624 U (Pantone U)) |

Difference: ΔE = 14.3

The use of ascorbic acid instead of cysteine results in a slightly less dark, red and more yellowish result when dyed at 37° C. The two color results differ from one another by ΔE=14.3.

TABLE 5
Comparison between treatment according to the invention with indican/cellulase
and cysteine and isatin at 20° C. and non-inventive treatment
with indican/cellulase and ascorbic acid and isatin at 20° C.
immediately after rinsing and blow-drying (L*, a*, b* values are shown)

| Color result according to the invention (day 0) | | | Non-inventive color result (day 0) | | |
|---|---|---|---|---|---|
| 59.7 | 11.4 | 15.5 | 57.6 | 13.3 | 202 |

| Cinnamon, caramel brown, Renwick rose- | Light topaz ocher (2318 U (Pantone U)) |
|---|---|
| beige (4655 U (Pantone U)) | |

Difference: ΔE = 5.5

The difference between the color results according to the invention and those not according to the invention is smaller when dyeing at 20° C. than when dyeing at 37° C.: ΔE=5.5 instead of 14.3. The strand treated with cysteine (according to the invention) is a little lighter, less red and less yellowish than the strand not dyed according to the invention.

1. An agent for non-oxidatively dyeing keratin fibers, the agent comprising:
at least one indican-containing component which that has been pretreated at a temperature of from 60° C. to 200° C.;
an enzyme mixture comprising at least one endo-1,4-beta-glucanase (EC 3.2.1.4), at least one beta-glucosidase (EC 3.2.1.21), and at least one exo-1,4-beta-D-glucanase (EC 3.2.1.91);
cysteine;
isatin; and
water.

2. The agent of claim 1, wherein the indican-containing component is selected from the group consisting of crushed parts of an indigo- or indirubin-producing plant, an indican-containing extract of an indigo- or indirubin-producing plant, and any mixture thereof.

3. The agent of claim 2, wherein the indican-containing extract from the indigo- or indirubin-producing plant is extracted using an extraction agent selected from the group consisting of water, C₁-C₄ alkanols, C₂-C₄ polyols, ethanol, isopropanol, n-propanol, ethylene glycol, 1,2-propanediol, glycerin, 1,3-butylene glycol, and any mixture thereof.

4. The agent of claim 2, wherein the indigo- or indirubin-producing plant is selected from at least one species of the group consisting of Indigofera, Isatis, Persicaria, Wrightia, Calanthe, Baphicacanthus, and any combination thereof.

5. The agent of claim 1, wherein the at least one indican-containing component comprises indican present in an amount of from 0.01-6 wt. %, based on the total weight of the at least one indican-containing component.

6. The agent of claim 1, wherein the at least one indican-containing component is pretreated by:
heating the at least one indican-containing component in a solvent, and
drying by steam or by dry heating the heated at least one indican-containing component in the solvent.

7. The agent of claim 6, wherein the extracted indican is separated from the solvent and solid plant parts such that the at least one indican-containing is in powder form.

8. The agent of claim 1, wherein:
the at least one endo-1,4-beta-glucanase (EC 3.2.1.4) is present in an amount of from 20-35 wt. %, based on the total weight of the enzyme mixture,
the at least one beta-glucosidase (EC 3.2.1.21) is present in an amount of from 15-25 wt. %, based on the total weight of the enzyme mixture, and
the at least one exo-1,4-beta-D-glucanase (EC 3.2.1.91) is present in an amount of from 25-35 wt. %, based on the total weight of the enzyme mixture.

9. The agent of claim 1, wherein the enzyme mixture is present in an amount of from 0.0001-1 wt. %, based on the total weight of the agent.

10. The agent of claim 1, wherein the cysteine is present in an amount of from 0.1-5.0 wt. %, based on the total weight of the agent.

11. The agent of claim 1, wherein the isatin is present in an amount of from 0.01-1.0 wt. %, based on the total weight of the agent.

12. The agent of claim 1, wherein the water is present in an amount of from 19.9-95.0 wt. %, based on the total weight of the agent.

13. The agent of claim 1, wherein a pH of the agent is from 2.0 to less than 10.0, measured at 20° C.

14. A kit for non-oxidative dyeing of keratin fibers, the kit comprising:
a first compartment comprising a composition comprising at least one indican-containing component that has been pretreated at a temperature of from 60° C. to 200° C.;
a second compartment comprising an aqueous composition comprising an enzyme mixture comprising at least one endo-1,4-beta-glucanase (EC 3.2.1.4), at least one beta-glucosidase (EC 3.2.1.21), and at least one exo-1,4-beta-D-glucanase (EC 3.2.1.91); and
a third compartment,
wherein at least one of the first compartment, the second compartment, or the third compartment comprises cysteine, and
wherein at least one of the first compartment, the second compartment, or the third compartment comprises isatin.

15. A method for non-oxidatively dyeing keratin fibers, the method comprising:
providing a composition comprising at least one indican-containing component that has been pretreated at a temperature of from 60° C. to 200° C.;
providing an aqueous composition comprising an enzyme mixture comprising at least one endo-1,4-beta-glucanase (EC 3.2.1.4), at least one beta-glucosidase (EC 3.2.1.21), and at least one exo-1,4-beta-D-glucanase (EC 3.2.1.91), wherein at least one of the composition comprising the at least one indican-containing component, the aqueous composition comprising the enzyme mixture, or a third component comprises cysteine, and wherein at least one of the composition comprising the at least one indican-containing component, the aqueous composition comprising the enzyme mixture, or the third component comprises isatin;
mixing the composition comprising the at least one indican-containing component and the aqueous composition comprising the enzyme mixture to produce a ready-to-use dye;
applying the ready-to-use dye to the keratin fibers;
maintaining the ready-to-use dye in the keratin fibers for a time of from 30 seconds to 60 minutes and at a temperature of from 25-60° C.; and
rinsing the keratin fibers.

16. The agent of claim 4, wherein the indigo- or indirubin-producing plant is selected from at least one species of Indigofera tinctoria.

17. The agent of claim 6, wherein the heated at least one indican-containing component in the solvent is dried for 5 to 10 minutes.

18. The agent of claim 9, wherein the enzyme mixture is present in an amount of from 0.01-0.03 wt. %, based on the total weight of the agent.

19. The kit of claim 14, wherein the first compartment, the second compartment, and the third compartment are physically separated from one another.

20. The method of claim 15, wherein the ready-to-use dye is maintained in the keratin fibers at a temperature of from 35-40° C.
